(19) United States
(12) Patent Application Publication
(10) Pub. No.: US 2025/0292897 A1
(43) Pub. Date: Sep. 18, 2025

(54) Systems and Methods for Proximity Based Data Transfer

(71) Applicant: Spacelabs Holdings, Inc., Snoqualmie, WA (US)

(72) Inventors: Zachary Orlowski, Penfield, NY (US); William P. Brown, Issaquah, WA (US); Jeffrey Wang, Duvall, WA (US)

(21) Appl. No.: 19/078,825

(22) Filed: Mar. 13, 2025

Related U.S. Application Data
(60) Provisional application No. 63/565,330, filed on Mar. 14, 2024.

(51) Int. Cl. G16H 40/60; G16H 10/65; H04W 4/38; H04W 4/80

(52) U.S. Cl. CPC G16H 40/60; G16H 10/65; H04W 4/38; H04W 4/80

(57) ABSTRACT

Medical systems include convenient and secure methods and systems for transferring medical data associated with a unique identity (ID) of a patient from a first patient monitoring device to a second patient monitoring device. The medical data associated with the unique ID includes physiological data of the patient monitored by the first patient monitoring device. At least one computing device in communication with the first patient monitoring device and the second patient monitoring device receives the data associated with the unique ID from the first patient monitoring device, receives the unique ID from the second patient monitoring device, receives a request to transfer the data associated with the unique ID to the second patient monitoring device, and causes the data associated with the unique ID to be transmitted to the second patient monitoring device.

### CROSS-REFERENCE

The present application relies on U.S. Patent Provisional Application No. 63/565,330, titled “System and Method for Proximity-Based Data Transfer”, and filed on Mar. 14, 2024, for priority, which is herein incorporated by reference in its entirety.

### FIELD

The present specification relates to medical systems, and in particular, to data transfer between medical monitors located proximate to one another in a hospital environment.

### BACKGROUND

Typically, hospitals have a plurality of patient monitors for monitoring and recording a multitude of physiological parameters of patients within the hospital. A patient is often monitored via more than one patient monitoring device in succession. When the patient moves on from a first patient monitoring device to a second, all data corresponding to the patient recorded by the first patient monitoring device is also desired to be transferred from the first patient monitoring device to the second patient monitoring device.

In some known scenarios, each patient is provided with a unique patient identifier (PID) and each patient monitoring device has a unique device identifier (DID). Various methods are used for associating a patient with a patient monitoring device. Patient data, along with the corresponding PID, may be input into a patient monitoring device manually by a clinician when the patient is admitted to the hospital for the first time. This method of patient association is prone to human errors and may lead to incorrect patient-device association, which, in turn, would lead to inaccuracies in the patient's medical records.

In some cases, wrist bands with bar codes containing information regarding a patient being admitted to a hospital are provided. The patient's wristbands may be scanned using a patient monitoring device to form an association with the device. In some situations, known mobile or computer applications, which scan both the patient's barcode and monitoring device may also be used for associating a patient with a patient monitoring device. These methods avoid manual error, but may be time consuming as they involve the steps of opening a menu system of the monitoring device, selecting the patient ID manually, and then scanning the wristband. In another method of associating a patient with a patient monitoring device, a clinician may select a device associated with a patient via an electronic medical record (EMR) system, which is also prone to human errors and risks of inaccurate selection by the clinician.

Reducing cognitive load, time on task and chance of error are large focal points in any health delivery organization. Thus, there is a need for a system and method for patient data transfer from one device to another within a hospital environment that is quick and efficient, yet accurate. There is also a need for a system and method that allows a clinician to simply transfer the patient association from one monitoring device to another monitoring device without any extra button presses, bar code scans, or manual entries. Further, there is a need for an efficient data transfer method that reduces cognitive load on the clinician, reduces the chance of errors of manual entries, and reduces the time taken to perform the data transfer task.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a system adapted to transferring medical data associated with unique identity (ID) data of a patient between monitoring devices, the system comprising: a first patient monitoring device of said monitoring devices associated with the unique ID data, wherein the first patient monitoring device is configured with a first wireless data receiver and a first wireless data transmitter, and wherein the first patient monitoring device is adapted to monitor the medical data of the patient; a second patient monitoring device of said monitoring devices configured with a second wireless data receiver and a second wireless data transmitter, wherein the first patient monitoring device is adapted to wirelessly transmit the unique ID data to the second patient monitoring device and wherein the second patient monitoring device is configured to wirelessly receive the unique ID data from the first patient monitoring device; at least one database for storing the medical data associated with the unique ID; at least one computing device in data communication with each of the first patient monitoring device, the second patient monitoring device, and the at least one database, wherein the at least one computing device is configured to execute a plurality of programmatic instructions that, when executed, cause the at least one computing device to: receive the medical data monitored by the first patient monitoring device; transmit the received medical data to the at least one database; receive from the first patient monitoring device a request to transfer the medical data associated with the unique ID to the second patient monitoring device; receive from the second patient monitoring device the unique ID; and in response to receiving request to transfer the medical data from the first patient monitoring device and to receiving the unique ID from the second patient monitoring device, cause the medical data associated with the unique ID to be transmitted to the second patient monitoring device.

Optionally, when the plurality of programmatic instructions are executed, at least one computing device is configured to refrain from transmitting the received medical data to the second patient monitoring device unless the at least one computing device first receives said request from the first patient monitoring device to transfer the medical data associated with the unique ID to the second patient monitoring device and receives said unique ID data from the second patient monitoring device.

Optionally, the first patient monitoring device is configured to wirelessly directly transmit the unique ID to the second patient monitoring device upon physical contact with the second patient monitoring device. Optionally, the first patient monitoring device is configured to only directly transmit the unique ID to the second patient monitoring device upon physical contact with the second patient monitoring device and not to transmit the medical data directly to the second patient monitoring device.

Optionally, the first patient monitoring device is configured to wirelessly directly transmit, and the second patient monitoring device is configured to wirelessly directly receive, the unique ID using near field communication. Optionally, the first patient monitoring device and the second patient monitoring device are configured to activate near field communication only by physical contact between the first patient monitoring device and the second patient monitoring device.

Optionally, the medical data associated with the unique ID comprises demographic data of the patient and physiological data of the patient. Optionally, the at least one computing device is adapted to transfer the demographic data associated with the unique ID to the second patient monitoring device only after the at least one computing device receives said request from the first patient monitoring device to transfer the medical data associated with the unique ID to the second patient monitoring device and receives said unique ID data from the second patient monitoring device.

Optionally, the first patient monitoring device is a multi-parameter patient monitor configured to monitor a plurality of patient parameters of a neonate patient, a pediatric patient or adult patient. Optionally, the plurality of patient parameters comprises one or more of ECG, heart rate, respirations, blood oxygenation, non-invasive blood pressure, invasive blood pressure, non-invasive temperature, invasive temperature, and cardiac output.

Optionally, the second patient monitoring device is a multi-parameter patient monitor configured to monitor a plurality of patient parameters of a neonate patient, a pediatric patient or adult patient. Optionally, the plurality of patient parameters comprises one or more of ECG, heart rate, respirations, blood oxygenation, non-invasive blood pressure, invasive blood pressure, non-invasive temperature, invasive temperature, and cardiac output.

The present specification also discloses a method for transferring medical data associated with a unique identity (ID) of a patient from a first patient monitoring device to a second patient monitoring device using at least one computing device remotely located from, and in data communication with, both the first patient monitoring device and the second patient monitoring device, wherein the medical data associated with the unique ID comprises physiological data of the patient monitored by the first patient monitoring device, said method comprising: using the first patient monitoring device, transmitting the medical data associated with the unique ID to the at least one computing device; receiving, at the at least one computing device, the medical data from the first patient monitoring device; using the first patient monitoring device, directly transmitting the unique ID from the first patient monitoring device to the second patient monitoring device; receiving, at the second patient monitoring device, the unique ID; using the first patient monitoring device, transmitting to the at least one computing device a request to transfer the medical data to the second patient monitoring device; using the second patient monitoring device, transmitting to the at least one computing device the unique ID; using the at least one computing device, causing the medical data associated with the unique ID to be transmitted to the second patient monitoring device.

Optionally, the method further comprises, using the at least one computing device, causing the medical data to be stored in at least one database. Optionally, the method further comprises, using the at least one computing device, causing the medical data to be transmitted to the second patient monitoring device from the at least one database.

Optionally, the first patient monitoring device is not configured to, permitted to, or enabled to directly transmit the medical data to the second patient monitoring device.

Optionally, causing the medical data to be transmitted to the second patient monitoring device using the at least one computing device only occurs after the at least one computing device receives said request from the first patient monitoring device to transfer the medical data to the second patient monitoring device and receives said unique ID data from the second patient monitoring device.

Optionally, the first patient monitoring device directly transmits the unique ID to the second patient monitoring device only upon physical contact with the second patient monitoring device.

Optionally, the first patient monitoring device directly transmits, and the second patient monitoring device directly receives, the unique ID using near field communication. Optionally, the method further comprises activating said near field communication only by physical contact between the first patient monitoring device and the second patient monitoring device.

Optionally, the first patient monitoring device and the second patient monitoring device are multi-parameter patient monitors configured to monitor a plurality of patient parameters of a neonate patient, a pediatric patient or adult patient. Optionally, the plurality of patient parameters comprises one or more of ECG, heart rate, respirations, blood oxygenation, non-invasive blood pressure, invasive blood pressure, non-invasive temperature, invasive temperature, and cardiac output.

The present specification also discloses a non-transient computer readable medium comprising a plurality of programmatic instructions that enable a transfer of medical data associated with a unique identity (ID) of a patient from a first patient monitoring device to a second patient monitoring device using at least one computing device remotely located from, and in data communication with, both the first patient monitoring device and the second patient monitoring device, wherein the medical data associated with the unique ID comprises physiological data of the patient monitored by the first patient monitoring device, and wherein executing the plurality of programmatic instructions causes: the medical data associated with the unique ID to be transmitted from the first patient monitoring device to the at least one computing device; the at least one computing device to receive the medical data from the first patient monitoring device; the unique ID to be directly transmitted from the first patient monitoring device to the second patient monitoring device; the second patient monitoring device to receive the unique ID; the first patient monitoring device to transmit to the at least one computing device a request to transfer the medical data to the second patient monitoring device; the second patient monitoring device to transmit to the at least one computing device the unique ID; and the medical data associated with the unique ID to be transmitted to the second patient monitoring device from the at least one computing device.

In some embodiments, the present specification also discloses a system for transferring data corresponding to a patient from a first patient monitor to a second patient monitor, wherein said data comprises a unique patient ID (PID), wherein the first patient monitor has data corresponding to the patient along with the PID stored locally on the first patient monitor, and wherein the first patient monitor and the second patient monitor each comprise a wireless data receiver and a wireless data transmitter, the system comprising: a server coupled with each of the first patient monitoring device and the second patient monitoring device, wherein the server is configured to a plurality of processing steps and wherein the plurality of processing steps comprise: receiving the patient's data from the first patient monitor; transmitting the patient's data to one or more second servers, wherein the one or more second servers store medical records associated with the patient; receiving a request to transfer data corresponding to the patient's PID from the first patient monitor to the second patient monitor; causing the second patient monitor to display a request for confirmation of the data transfer; receiving a response to the request for confirmation from a user of the second patient monitor; enabling a wireless transfer of the patient's PID from the first patient monitor to the second patient monitor; receiving the patient's PID from the second patient monitor; and causing medical records associated with the patient to be transmitted from the one or more second servers to the second patient monitor.

Optionally, the one or more second servers comprise an electronic medical record (EMR) system.

Optionally, the server transfers the patient's data and PID received from the first patient monitor to the EMR system for updating the medical records associated with the patient in the EMR system.

Optionally, the first patient monitor and the second patient monitor are configured to use near field communication (NFC).

Optionally, the wireless transfer of the patient's PID from the first patient monitor to the second patient monitor occurs when the second patient monitor is tapped on by the first patient monitor.

Optionally, the server only executes each of said plurality of processing steps after the user places the second patient monitor in physical proximity to the first patient monitor.

Optionally, each of the first patient monitor and second patient monitor are provided with a unique device identifier (DID).

Optionally, each of the first patient monitor and second patient monitor is a multi-parameter patient monitor configured to be used on neonate, pediatric and adult patients for recording and monitoring a plurality of patient parameters.

Optionally, the plurality of patient parameters comprises one or more of ECG, heart rate, respirations, blood oxygenation, non-invasive blood pressure, invasive blood pressure, non-invasive temperature, invasive temperature, and cardiac output.

In some embodiments, the present specification discloses a method for transferring data corresponding to a patient from a first patient monitoring device to a second patient monitoring device, said method comprising: associating a patient having a unique first patient ID (PID) with the first patient monitoring device; monitoring and recording physiological data corresponding to the patient having the unique first PID by the first patient monitoring device; sending the recorded physiological data to a system server and to an electronic medical record (EMR) system for updating medical records of the patient; placing the first patient monitoring device in close proximity to a second patient monitoring device for transferring medical data of the patient having the unique first PID, wherein the first and the second patient monitoring devices are NFC (near field communication) enabled; displaying a message on the second patient monitoring device requesting confirmation from a clinician for transferring medical data of the patient having the unique first PID from the first patient monitoring device to the second patient monitoring device; receiving the confirmation on the second patient monitoring device; transferring the unique first PID to the second patient monitoring device from the first patient monitoring device; communicating the first unique PID to the system server by the second patient monitoring device; and transferring patient data corresponding to the unique first PID to the second monitoring device by the system server.

Optionally, the method further comprises monitoring and recording specific medical data corresponding to the patient having the unique first PID by the second patient monitoring device; and communicating the recorded patient data corresponding to the patient having the first unique PID to the system server by the second patient monitoring device for updating the patient's medical records.

Optionally, the method further comprises communicating the updated patient record corresponding to the patient having the first unique PID by the system server with the EMR system for updating the patient's medical records in the EMR system.

Optionally, the clinician manually enters at least a portion of the data corresponding to a patient into the first patient monitor for associating the patient with a unique PID.

Optionally, the data for associating the patient with a unique PID is received by the first monitoring device from the EMR system as an admit message (ADT) via the system server.

Optionally, the method further comprises transferring patient data corresponding to the unique first PID to the second monitoring device by the system server comprises transferring demographic and physiological patient data corresponding to the unique first PID to the second monitoring device.

Optionally, each of the first and second patient monitoring devices are provided with a unique device identifier (DID).

Optionally, sending the recorded physiological data to a system server and to an electronic medical record (EMR) system for updating medical records of the patient comprises sending a DID of the first patient monitoring device to the EMR system.

Optionally, each of the first and second patient monitoring devices is a multi-parameter patient monitor that is intended to be used on neonate, pediatric and adult patients for recording and monitoring a plurality of patient parameters.

Optionally, the system server is a predefined Enterprise System which stores data, interfaces with a hospital information system and the EMR system.

Optionally, the wireless transfer of the patient's PID from the first patient monitor to the second patient monitor occurs when the second patient monitor is tapped on by the first patient monitor.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a block diagram illustrating a system for proximity-based patient data transfer, in accordance with an embodiment of the present specification;

FIG. 1B illustrates a patient monitor for use in the system for proximity-based patient data transfer, in accordance with an embodiment of the present specification;

FIG. 1C illustrates an exemplary screen display of the second monitoring device for requesting data transfer confirmation from a clinician, in accordance with an embodiment of the present specification;

FIG. 2 is a flowchart illustrating a method for proximity-based patient data transfer, in accordance with an embodiment of the present specification; and

FIG. 3 is a flowchart illustrating an exemplary use case of the system and method of the present specification.

### DETAILED DESCRIPTION

In embodiments the present specification provides a system and method for proximity-based transfer of patient data in a clinical/hospital setting, from a first patient monitor to another patient monitor. In an embodiment, near field communication (NFC) technology is used to enable the proximity-based data transfer.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

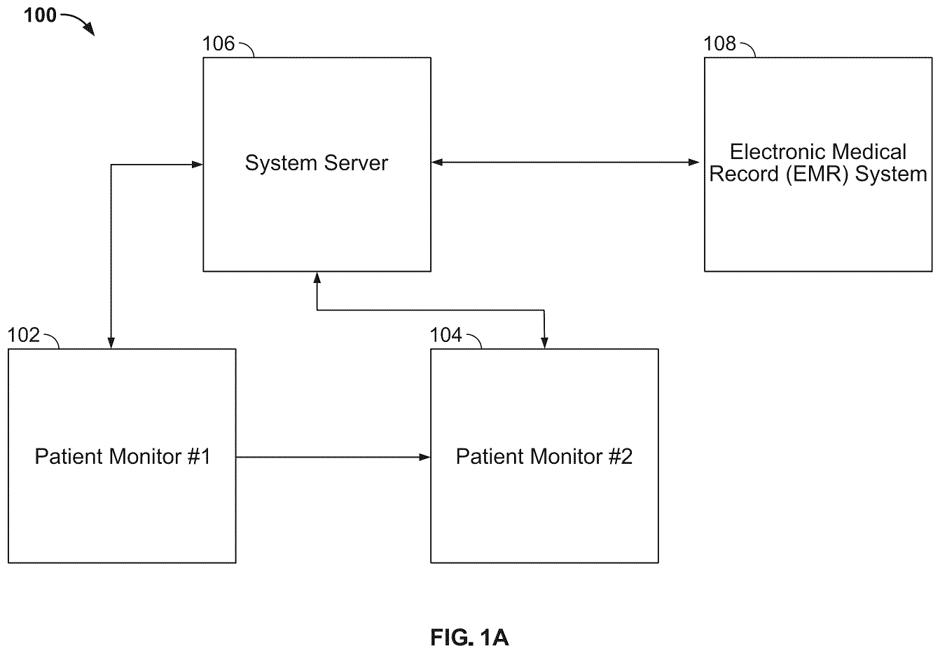

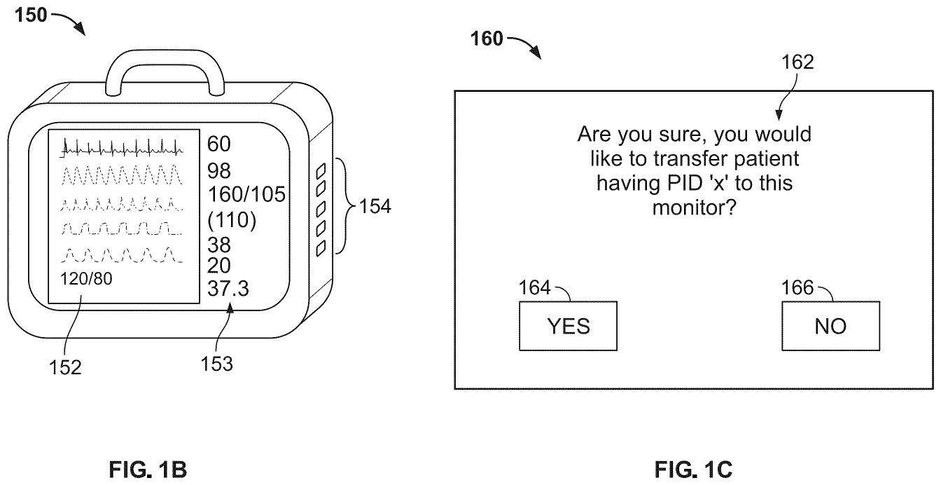

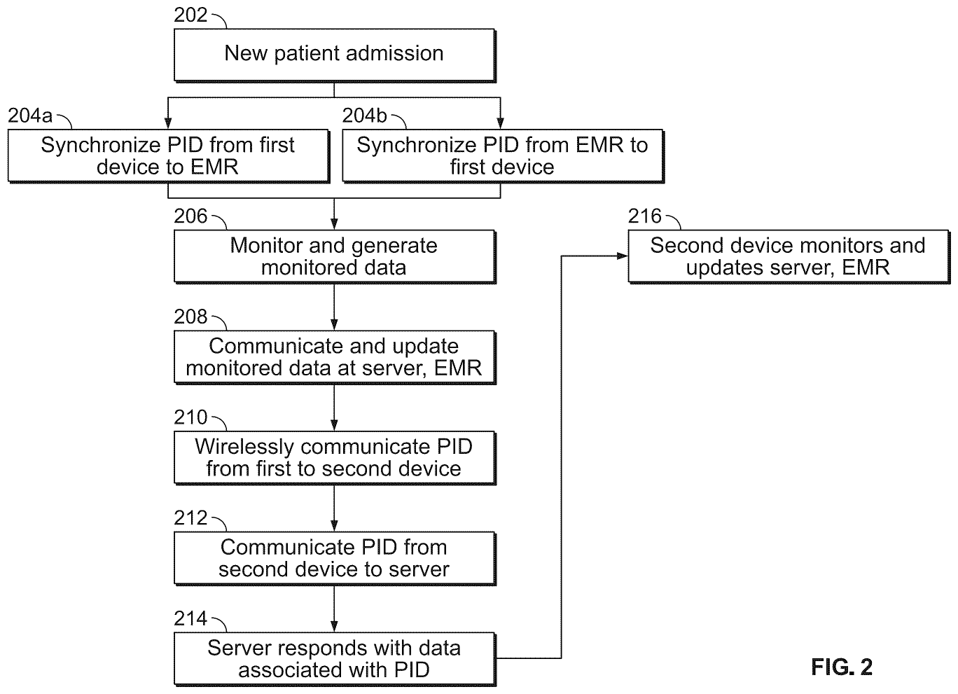

FIG. 1A is a block diagram illustrating a system that is configured to transfer patient data, in accordance with some embodiments of the present specification. System 100 comprises a first patient monitoring device 102 and a second patient monitoring device 104, both of which are in data communication with a system server 106. System server 106 is also in communication with at least one database. The database is optionally part of an electronic patient record (EMR) system 108. The at least one database is configured to store data corresponding to a plurality of patients. The data for each patient is identified by a unique ID assigned to the corresponding patient. Hereinafter, the unique ID is termed as patient ID (PID). In embodiments, patient monitoring devices 102, 104 include and are not limited to telemetry monitors and wireless single parameter measurement devices such as, but not limited to, SpO2 sensors, and wireless NIBP (Non-Invasive Blood Pressure) devices.

FIG. 1B is an illustration of an exemplary patient monitoring device 150. Device 150 includes a display 152 configured to display the various physiological parameters 153 that are monitored by device 150. Display 152 is for representational purposes only. The actual display 152 may vary for different embodiments and applications of the present specification. Additionally, an interface 154 provides a combination of buttons that are configured to control the display and operated by a user/clinician to manipulate the parameters shown on the display, to select options offered by device 150, to input parameters such as details of a patient that is being monitored by the device, among other options and controls. In embodiments, patient monitor 150 is a single parameter or multi-parameter patient monitor that is intended to be used on neonate, pediatric and adult patients for recording and monitoring a plurality of medical data corresponding to patient parameters 153 such as, but not limited to, ECG, heart rate, respiration rate, blood oxygenation, non-invasive blood pressure, invasive blood pressure, non-invasive temperature, invasive temperature, and cardiac output. In embodiments, patient monitor 150 is configured to send the recorded plurality of patient data to a predefined server such as, but not limited to, a predefined enterprise system (server 106) which stores data, interfaces with the hospital information system/electronic medical record, and performs other functions (such as, but not limited to printing and exporting data in various forms). Patient monitor 150 is configured to send data such as a patient's clinical/physiological and demographics data as well as technical configurations that may have been received from an external system. The technical configurations may include information such as, and not limited to, time, date, IP addresses, wireless communications settings, among other types of information. In embodiments, the technical configurations are configured on system server 106 (see FIG. 1A). In some embodiments, technical configurations include information associated with patient monitor 150.

Each of the first patient monitoring device 102 and second patient monitoring device 104 can monitor at least one of: heart rate, blood pressure, skin temperature, respiratory rate, electrocardiogram, cardiac output, non-invasive body temperature, invasive body temperature, skin conductance, blood glucose, blood oxygen saturation, non-invasive blood pressure, invasive blood pressure, EEG and EMG, among other types of physiological data of a patient. Each monitoring device 102, 104 is configured to monitor data of a patient and associate the monitored data of the patient with the patient's PID. The PID may include a medical record number (MRN), a social security number (SSN), or any other version of unique patient identifiers. In an embodiment, each of first device 102 and second device 104 are located in a clinic/hospital environment and are associated with a unique device ID (DID).

It should be noted that each component, processor, patient monitor, and/or device described herein is configured to perform the functions and communications described herein. In embodiments, each patient monitoring device 102, 104, server 106, and/or EMR system 108 is capable of executing programmatic instructions. It should further be appreciated that each device and monitoring system have wireless and wired receivers and transmitters capable of sending and transmitting data, at least one processor capable of processing programmatic instructions, memory capable of storing programmatic instructions, and software comprised of a plurality of programmatic instructions for performing the processes described herein.

In embodiments, the data associated with a PID includes demographic data and physiological data, where the latter type of data is specifically collected by the patient monitoring devices. Patient demographic data may include non-clinical or non-medical data of the patient, such as, but not limited to, name, age, gender, date of birth, address, medical history, and insurance information. Physiological data includes medical or clinical data of the patient such as, for example, heart rate, blood pressure, skin temperature, respiratory rate, electrocardiogram, cardiac output, non-invasive body temperature, invasive body temperature, skin conductance, blood glucose, blood oxygen saturation, non-invasive blood pressure, invasive blood pressure, EEG and EMG, among other types of physiological data. The data associated with a PID includes both demographic data and physiological data corresponding to the patient who is allocated the PID.

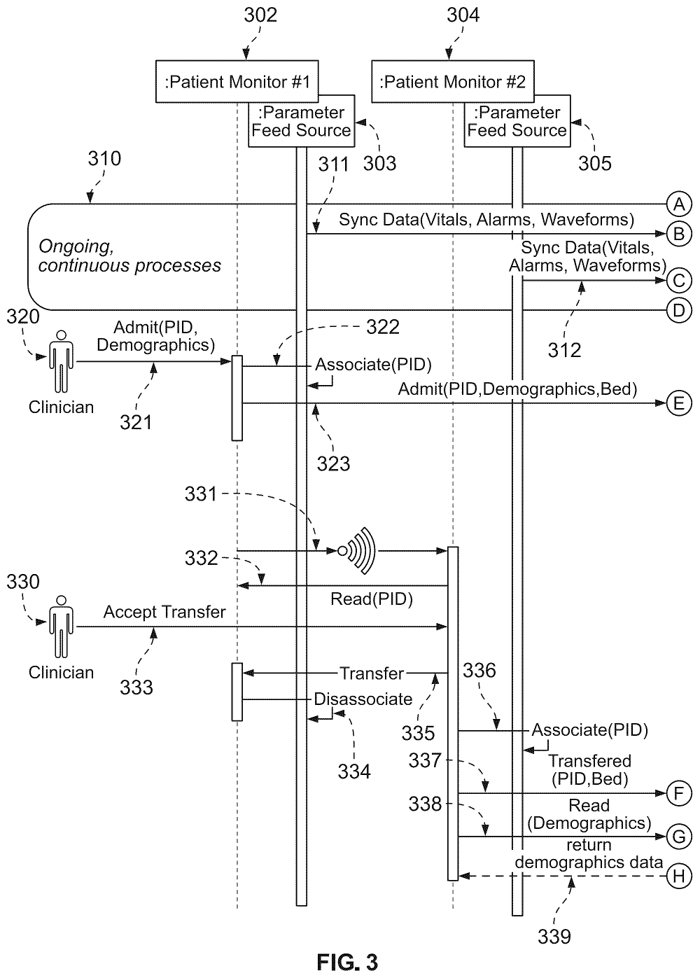

FIG. 2 is a flow chart illustrating steps of an exemplary process for transferring data from first patient monitoring device 102 (first device 102) to second patient monitoring device 104 (second device 104), in accordance with some embodiments of the present specification. Referring simultaneously to FIGS. 1A and 2, at step 202, a patient is admitted to a medical facility such as a clinic, a hospital, or a home. During the admission, the patient is assigned to and/or associated with a patient monitoring device, for example first device 102, to monitor the patient's vitals and any other physiological parameters that may be warranted. In one embodiment, at step 204a, patient data is input into first device 102, using a user interface that is embedded within device 102, where the patient data may include the patient's demographic data and a PID. First device 102, which is in communication with server 106, communicates the patient data input by the user to server 106. The patient data includes the PID of the patient. In embodiments, first device 102 may be identified using a device ID (DID) that informs server 106 about the source of the patient information that is provided to it. Server 106 further communicates the received data including the PID to EMR 108, thereby creating or updating the patient's medical records with information about the patient's admission, among other parameters, provided through first device 102.

In an alternate embodiment, at step 204b, EMR 108 is updated with information about the patient's admission to the medical facility. EMR 108 maintains medical records of the patient identified with the PID and communicates the information pertaining to the patient's admission to server 106. The information is communicated by EMR 108 in the form of an admit message (ADT). For example, EMR 108 may send an ADT message to server 106, stating that patient by the name of ‘John Smith’ having a PID ‘P1’ is admitted to a particular hospital. Server 106, in turn, communicates the ADT message to first device 102 which is set up and configured to monitor the patient. Therefore, using either 204a or 204b, first device 102, server 106 and EMR 108 are synchronized with the information pertaining to the patient identified with the corresponding PID.

At step 206, the physiological data of the patient is monitored by first device 102. Optionally, device 102 includes a memory or is attached to a local memory that stores the monitored physiological data and the demographic data of the patient being monitored. The data associated with the PID comprises the demographic data and the physiological data. The physiological data may include historic data maintained by EMR 108, as well as the data monitored by first device 102.

At step 208, first device 102 communicates the monitored physiological data associated with the PID to server 106, which is also reflected at EMR 108 through server 106. The monitored physiological data may be communicated to server 106 and updated at EMR 108 either in real time, at intervals, or each time when first device 102 is brought online to activate the communication between device 102 and server 106.

There may be instances where the patient data that is generated from the first device 102 from the patient monitoring is required to be transferred to a different patient monitoring device such as second device 104. An example scenario is when a patient is transferred from one medical facility to another, or from one room with first device 102 to another room with second device 104. As a result of the transfer, the patient is attached to second patient monitoring device 104, which does not have prior information stored with or monitored by first device 102. At step 210, the data transfer from first device 102 to second device 104 is enabled by wirelessly transmitting the PID. In an embodiment, wireless transmission of PID is enabled by using the near field communication (NFC) technology. Accordingly, first device 102 is brought in proximity to second device 104. The two devices are optionally tapped against each other to support and enable the NFC communication. The tapping action results in a prompt, which is displayed on a screen of second device 104, asking the user to confirm transfer of PID from first device 102 to second device 104. FIG. 1C illustrates an exemplary prompt 160 displayed by second device 104. In embodiments, a text 162 with a confirmation message, such as “Are you sure, you would like to transfer patient having PID ‘x’ to this monitor?” is shown along with interactive response options “Yes” 164 and “No” 166. It is essential for the clinician to provide the confirmation prior to the system initiating data transfer between patient monitoring systems to eliminate any risk of inadvertent data transfers and associated diagnostic mistakes. The user may maintain a distance of approximately less than or equal to 5 cm while selecting option 164 (Yes) to initiate transfer of PID from first device 102 to second device 104. As stated earlier, NFC technology for proximity-based data transfer is used to transfer the patient's PID to second device 104. In one embodiment, either or both first and second device 102, 104 are configured with wireless receivers and transmitters that support NFC. In another embodiment, either one of or both first device 102 and second device 104, is/are coupled to a peripheral device (memory/computing device such as, but not limited to, a USB device) carrying NFC technology. As is known, near-field communication (NFC) is a short-range wireless connectivity technology that uses magnetic field induction to enable communication between devices when the devices are in physical contact with one another or brought within a few centimeters of each other.

In one embodiment, first device 102 retains the demographic and physiological data of the patient with the PID even after transferring the PID to second device 104. In another embodiment, the demographic and physiological data corresponding to the PID is erased from first device 102 after the transfer of the PID to second device 104 is complete. In embodiments, a confirmation message corresponding to the completion of transfer of the unique PID to second device 104 may be displayed on a screen of second device 104, or/and on a remote display coupled with second device 104, and server 106, or/and an application running on a clinician's mobile device that is coupled with second device 104 and/or to server 104.

At step 212, upon receiving a confirmation that the PID has been transferred, second device 104 communicates the PID to server 106, wherein the communication of the PID to server 106 is a part of a request from second device 104 to obtain data associated with the PID of the patient. At step 214, server 106 recognizes and notes that the patient with the communicated PID is now being monitored by second device 104, and not by first device 102. In an embodiment, second device 104 is also identified by a unique device ID (DID) that is updated with server 106. Server 106 further communicates the request for data associated with the PID to a database associated with EMR 108. EMR 108 responds by sharing data associated with PID to server 106. Optionally, server 106 also includes a local database that is additionally updated with the status of PID. The database associated with server 106 stores data associated with the PID.

Therefore, at step 214, server 106 responds to the request received from second device 104 by sending data associated with the PID. The data may include at least the demographic data of the patient with the given PID, or may include both the demographic data as well as the historic physiological data of the patient. In some embodiments, the second device 104 receives the demographic data of the patient. The user/clinician may generate a separate request for the entirety of or a part of historic physiological data, which is then obtained by server 106 and communicated to second device 104. For example, in a scenario, the second device 104 that is now associated with the PID, receives an input from a user (such as a clinician) to view an ECG waveform associated with the PID. In the given example, server 106 receives a request from second device 104 to view the waveform, and then server 106 responds by sending the requested waveform associated with the PID, to second device 104. In this example, second device 104 does not have to store physiological information associated with the PID. Therefore, second device 104 is configured to receive physiological data from server 106 on demand. In either scenario (storage of information associated with the PID or information on-demand), second device 104 is updated with patient information of the given PID.

At step 216, second device 104 now monitors the patient and generates physiological data which is communicated to and updated with server 106 and EMR 108. In an embodiment, EMR 108 is ‘device centric’, wherein a patient's medical record is stored as corresponding to a device having a unique DID. In such cases, first device 102 sends the patient's data to server 106 wherein the patient's data is identifiable by and/or associated with the DID of first device 102. Server 106 in turn communicates the DID to EMR 108 for updating the data associated with the PID.

In another embodiment, EMR 108 is ‘location centric’, wherein a patient's medical record is stored corresponding to a location/bed within a hospital where the patient with the unique PID is located/placed. In such cases, first device 102 sends the patient's data to server 106 wherein the patient's data is identifiable by and/or associated with a location/bed ID of the patient. Server 106 in turn communicates the location/bed ID to EMR 108 for updating the data associated with the PID.

The systems and methods of the present specification provide proximity-based data transfer which is simple to execute by a clinician and takes minimal time. The clinician is only required to “tap” one patient monitoring device to another to trigger a pop up that the clinician then only needs to click to confirm the data transfer. Hence, the systems and methods of the present specification allows a clinician to transfer data associated with a patient by simply bringing two separate monitors within close proximity of each other.

In one embodiment, the unique patient ID and/or the medical data is transferred directly from the first patient monitoring device to the second patient monitoring device without first being stored in another computing device's read only memory. In another embodiment, the unique patient ID and/or the medical data is transferred directly from the first patient monitoring device to the second patient monitoring device without first being stored in another computing device's read only memory. or random access memory. In yet another embodiment, the unique patient ID and/or the medical data is transferred directly from the first patient monitoring device to the second patient monitoring device by first being stored in another computing device's read only memory and/or random access memory.

In another embodiment, the unique patient ID and/or the medical data is transferred directly from the first patient monitoring device to the second patient monitoring device upon physical contact with the second patient monitoring device without first being stored in another computing device's read only memory and/or random access memory.

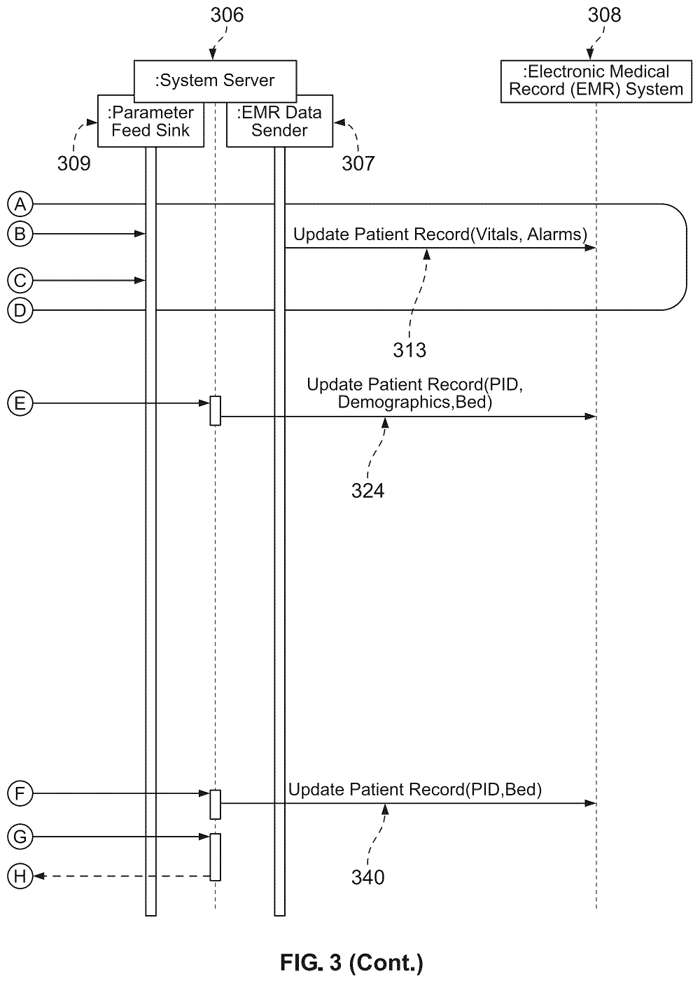

FIG. 3 is a flowchart illustrating an exemplary use case of the system and method of the present specification. The flowchart illustrates a sequence of information flow or communication of data between a first patient monitor 302, a second patient monitor 304, a system server 306, and an EMR system 308. The sequence of communication of data illustrated in FIG. 3 is for transfer of a patient associated with first patient monitor 302 to second patient monitor 304. Horizontal arrows are representative of the direction of flow of data between said devices, while the vertical direction, from top to bottom, indicates the progression of time (not shown to scale).

Ongoing and continuous processes 310 are now described, which indicate the information communicated on a continuous basis between the various components of the flow chart of FIG. 3. Patient monitors 302 and 304 continuously publish patient data which they are configured to monitor, to system server 306. The patient data monitored and generated by patient monitor 302 constitutes parameter feed source 303 and the patient data monitored and generated by patient monitor 304 constitutes parameter feed source 305. The patient data may include, among various patient parameters, a patient's vitals, alarms generated by the corresponding patient monitor, and waveforms associated with one or more physiological parameters monitored by the corresponding patient monitor. Patient monitor 302 is configured such that it synchronizes 311 parameter feed source 303 with system server 306 and similarly, patient monitor 304 is configured such that it synchronizes 312 parameter feed source 305 with system server 306; wherein parameter feed sources 303 and 305 are subscribed by system server 306 and are received at system server 306 in the form of a parameter feed sink 309. System server 306 is configured to store 307 the incoming parameter feed data provided by parameter feed source 303 and 305. Furthermore, the system server 306 is configured to process the incoming parameter feed data provided by parameter feed sources 303 and 305 and communicates 313 this data through a communication module 307 to EMR system 308. Processes 310 are continuous and ongoing for any patient associated with either patient monitor 303 or patient monitor 304. Processes 310 are not modified or altered as a result of transfer of a patient from one monitor to another.

Initially, a clinician 320 admits a patient and associates the patient with first monitor 302. Admit data 321, including a PID and optionally the patient's demographic data are entered by clinician 320 to the device of patient monitor 302 using a user interface of monitor 302. Patient monitor 302 accepts admit data 321 and updates/changes the association of parameter feed source 303 to be associated with the entered PID. From here onwards, all patient parameter data of monitor 302 is effectively stamped with the patient's PID so that the data is associated with that patient.

Patient monitor 302 then notifies system server 306 of the admission with a communication 323 that includes the PID, demographic data of the patient (if available), and any other patient-centric information such as and not limited to the bed number or ID of the bed allotted to the patient. System server 306 then notifies EMR 308 with an update 324 of the patient's record, so that EMR 308 further updates the patient record in its database accordingly.

In order to initiate transfer of patient data from patient monitor 302 to patient monitor 304, monitor 302, which is associated with patient PID, is brought in close physical proximity to monitor 304. In embodiments, a physical contact is made between monitors 302 and 306, such as by the action of tapping the two monitors briefly, and then keeping them in close proximity that ranges from 0 to 5 centimeters. Monitor 304 detects 331 the presence of monitor 302. Monitor 304 connects to monitor 302 with a secure communications protocol, such as and not limited to NFC, to determine if there is a patient to transfer. If there is a patient to transfer then monitor 304 reads 332 the PID, and optionally demographics, from monitor 302. Monitor 304 puts up a dialog (see FIG. 1C) that prompts the clinician 330 if they want to transfer the patient from monitor 302 to monitor 304. If clinician 330 responds with ‘yes’ 333 indicating acceptance of the need to transfer, then monitor 304 requests 335 monitor 302 to perform a transfer operation. Monitor 302 disassociates 334 parameter feed source 303 from the patient's PID. At this point in time, parameter data from monitor 302 to system server 306 is ‘anonymous’—not associated with any patient. In the absence of an association with a PID, parameter feed source 303 is not used to update 313 EMR 308.

Additionally, monitor 304 associates 336 its parameter feed source 305 with the patient's PID. Therefore, monitor 304 monitors parameter data 312 which is then used to update 313 the patient's record in EMR 308. Monitor 304 notifies 337 system server 306 of the transfer and system server 306 notifies 340 EMR 308. Monitor 304 sends a request 338 for the patient's demographic data, which it receives 339 from system Server 306 and uses it to display to clinician 320.

The above examples are merely illustrative of the applications of the systems and methods of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system adapted to transferring medical data associated with unique identity (ID) data of a patient between monitoring devices, the system comprising:
a first patient monitoring device of said monitoring devices associated with the unique ID data, wherein the first patient monitoring device is configured with a first wireless data receiver and a first wireless data transmitter, and wherein the first patient monitoring device is adapted to monitor the medical data of the patient;
a second patient monitoring device of said monitoring devices configured with a second wireless data receiver and a second wireless data transmitter, wherein the first patient monitoring device is adapted to wirelessly transmit the unique ID data to the second patient monitoring device and wherein the second patient monitoring device is configured to wirelessly receive the unique ID data from the first patient monitoring device;
at least one database for storing the medical data associated with the unique ID;
at least one computing device in data communication with each of the first patient monitoring device, the second patient monitoring device, and the at least one database, wherein the at least one computing device is configured to execute a plurality of programmatic instructions that, when executed, cause the at least one computing device to:
receive the medical data monitored by the first patient monitoring device;
transmit the received medical data to the at least one database;
receive from the first patient monitoring device a request to transfer the medical data associated with the unique ID to the second patient monitoring device;
receive from the second patient monitoring device the unique ID; and
in response to receiving request to transfer the medical data from the first patient monitoring device and to receiving the unique ID from the second patient monitoring device, cause the medical data associated with the unique ID to be transmitted to the second patient monitoring device.

2. The system of claim 1, wherein, when the plurality of programmatic instructions are executed, at least one computing device is configured to refrain from transmitting the received medical data to the second patient monitoring device unless the at least one computing device first receives said request from the first patient monitoring device to transfer the medical data associated with the unique ID to the second patient monitoring device and receives said unique ID data from the second patient monitoring device.

3. The system of claim 1, wherein the first patient monitoring device is configured to wirelessly directly transmit the unique ID to the second patient monitoring device upon physical contact with the second patient monitoring device.

4. The system of claim 3, wherein the first patient monitoring device is configured to only directly transmit the unique ID to the second patient monitoring device upon physical contact with the second patient monitoring device and not to transmit the medical data directly to the second patient monitoring device.

5. The system of claim 1, wherein the first patient monitoring device is configured to wirelessly directly transmit, and the second patient monitoring device is configured to wirelessly directly receive, the unique ID using near field communication.

6. The system of claim 5, wherein the first patient monitoring device and the second patient monitoring device are configured to activate near field communication only by physical contact between the first patient monitoring device and the second patient monitoring device.

7. The system of claim 1, wherein the medical data associated with the unique ID comprises demographic data of the patient and physiological data of the patient.

8. The system of claim 7, wherein the at least one computing device is adapted to transfer the demographic data associated with the unique ID to the second patient monitoring device only after the at least one computing device receives said request from the first patient monitoring device to transfer the medical data associated with the unique ID to the second patient monitoring device and receives said unique ID data from the second patient monitoring device.

9. The system of claim 1, wherein the first patient monitoring device is a multi-parameter patient monitor configured to monitor a plurality of patient parameters of a neonate patient, a pediatric patient or adult patient.

10. The system of claim 9, wherein the plurality of patient parameters comprises one or more of ECG, heart rate, respirations, blood oxygenation, non-invasive blood pressure, invasive blood pressure, non-invasive temperature, invasive temperature, and cardiac output.

11. The system of claim 1, wherein the second patient monitoring device is a multi-parameter patient monitor configured to monitor a plurality of patient parameters of a neonate patient, a pediatric patient or adult patient.

12. The system of claim 11, wherein the plurality of patient parameters comprises one or more of ECG, heart rate, respirations, blood oxygenation, non-invasive blood pressure, invasive blood pressure, non-invasive temperature, invasive temperature, and cardiac output.

13. A method for transferring medical data associated with a unique identity (ID) of a patient from a first patient monitoring device to a second patient monitoring device using at least one computing device remotely located from, and in data communication with, both the first patient monitoring device and the second patient monitoring device, wherein the medical data associated with the unique ID comprises physiological data of the patient monitored by the first patient monitoring device, said method comprising:
using the first patient monitoring device, transmitting the medical data associated with the unique ID to the at least one computing device;
receiving, at the at least one computing device, the medical data from the first patient monitoring device;
using the first patient monitoring device, directly transmitting the unique ID from the first patient monitoring device to the second patient monitoring device;
receiving, at the second patient monitoring device, the unique ID;
using the first patient monitoring device, transmitting to the at least one computing device a request to transfer the medical data to the second patient monitoring device;
using the second patient monitoring device, transmitting to the at least one computing device the unique ID;
using the at least one computing device, causing the medical data associated with the unique ID to be transmitted to the second patient monitoring device.

14. The method of claim 13, further comprising using the at least one computing device, causing the medical data to be stored in at least one database.

15. The method of claim 14, further comprising, using the at least one computing device, causing the medical data to be transmitted to the second patient monitoring device from the at least one database.

16. The method of claim 13, wherein the first patient monitoring device is not configured to, permitted to, or enabled to directly transmit the medical data to the second patient monitoring device.

17. The method of claim 13, wherein causing the medical data to be transmitted to the second patient monitoring device using the at least one computing device only occurs after the at least one computing device receives said request from the first patient monitoring device to transfer the medical data to the second patient monitoring device and receives said unique ID data from the second patient monitoring device.

18. The method of claim 13, wherein the first patient monitoring device directly transmits the unique ID to the second patient monitoring device only upon physical contact with the second patient monitoring device.

19. The method of claim 13, wherein the first patient monitoring device directly transmits, and the second patient monitoring device directly receives, the unique ID using near field communication.

20. The method of claim 19, further comprising activating said near field communication only by physical contact between the first patient monitoring device and the second patient monitoring device.

21. The method of claim 13, wherein the first patient monitoring device and the second patient monitoring device are multi-parameter patient monitors configured to monitor a plurality of patient parameters of a neonate patient, a pediatric patient or adult patient.

22. The method of claim 21, wherein the plurality of patient parameters comprises one or more of ECG, heart rate, respirations, blood oxygenation, non-invasive blood pressure, invasive blood pressure, non-invasive temperature, invasive temperature, and cardiac output.

23. A non-transient computer readable medium comprising a plurality of programmatic instructions that enable a transfer of medical data associated with a unique identity (ID) of a patient from a first patient monitoring device to a second patient monitoring device using at least one computing device remotely located from, and in data communication with, both the first patient monitoring device and the second patient monitoring device, wherein the medical data associated with the unique ID comprises physiological data of the patient monitored by the first patient monitoring device, and wherein executing the plurality of programmatic instructions causes:
the medical data associated with the unique ID to be transmitted from the first patient monitoring device to the at least one computing device;
the at least one computing device to receive the medical data from the first patient monitoring device;
the unique ID to be directly transmitted from the first patient monitoring device to the second patient monitoring device;
the second patient monitoring device to receive the unique ID;
the first patient monitoring device to transmit to the at least one computing device a request to transfer the medical data to the second patient monitoring device;
the second patient monitoring device to transmit to the at least one computing device the unique ID; and
the medical data associated with the unique ID to be transmitted to the second patient monitoring device from the at least one computing device.
